(19) United States
(12) Patent Application Publication
(10) Pub. No.: US 2021/0330719 A1
(43) Pub. Date: Oct. 28, 2021

(54) PROBIOTIC COMBINATION FOR TREATMENT OF INFLAMMATORY-RELATED GASTROINTESTINAL DISORDERS

(71) Applicant: Société des Produits Nestlé S.A., Vevey (CH)

(72) Inventors: Jalil BENYACOUB, Epalinges (CH); Stéphane DUBOUX, St-Prex (CH); Gail CZARNECKI-MAULDEN, Edwardsville, IL (US); Guénolée Eliane Marie PRIOULT, Bern (CH); Gabriela BERGONZELLI DEGONDA, BUSSIGNY (CH)

(21) Appl. No.: 17/273,635

(22) PCT Filed: Oct. 1, 2019
(86) PCT No.: PCT/EP2019/076498

§ 371 Date: Mar. 4, 2021

Related U.S. Application Data
(60) Provisional application No. 62/739,934, filed on Oct. 2, 2018.

(51) Int. Cl. A61K 35/745; A61P 29/00; A61P 1/14

(52) U.S. Cl. CPC A61K 35/745; A61P 1/14; A61P 29/00

(57) ABSTRACT

Disclosed is a probiotic combination comprising B. longum and B. lactis for the treatment or prevention of an inflammatory-related gastrointestinal disorder such as an inflammatory bowel disease.

The present invention relates to a combination of probiotics for the treatment or prophylaxis of gastrointestinal disorders, in particular inflammatory-related gastrointestinal disorders, and to compositions and methods employing the combination.

### BACKGROUND OF THE INVENTION

Inflammatory conditions of the gastrointestinal tract can cause severe discomfort and abdominal pain, and can severely impact quality of life. Typically, inflammatory-related gastrointestinal disorders, such as inflammatory bowel disease (IBD), manifest in symptoms of recurring bowel trouble, including diarrhea, abdominal cramping and/or pain, reduced appetite and associated weight loss, nausea, fever and fatigue. These symptoms can be episodic or persistent, and can lead to impairment of the ability to take up nutrients. This is particularly serious in children and adolescents, where it can lead to poor growth and a lack of weight gain. Crohn's disease and ulcerative colitis are the most common examples of inflammatory-related gastrointestinal disorders. The symptoms of IBD can be affected by diet and stress, and management of the symptoms by changes to lifestyle, nutrition and diet may offer some degree of relief. For example, symptoms can be exacerbated by the consumption of dairy, fatty foods, spicy foods, caffeine and alcohol, and in some cases, an excessive intake of dietary fibre. Crohn's disease is particularly troublesome and may cause severe abdominal pain and nutritional problems.

The prevalence of inflammatory-related gastrointestinal disorders such as IBD is increasing. Currently, IBD affects around 1.5 million people in the United States and 2.2 million in Europe. The causes of IBD is not known but a number of factors such as genetics, the immune system, intestinal epithelial barrier integrity and the environment are all understood to play a role.

Current drug treatments for inflammatory-related gastrointestinal disorders such as IBD include aminosalicylates, immune modifiers, antibiotics and corticosteroids. However, many patients will experience a high relapse rate after drug treatment, with many eventually requiring surgical intervention.

Inflammatory-related gastrointestinal disorders may also affect other mammals, for example companion mammals such as cat (e.g. feline IBD) and dogs (e.g. canine IBD). Dog and cats especially may suffer from inflammatory-related gastrointestinal disorders which may manifest as chronic diarrhoea (i.e. diarrhea persisting for 3 weeks or more). Inflammatory bowel conditions in animals may be in response to food (food-responsive diarrhea, diet-responsive diarrhea, food-responsive enteropathy), or may be of an unknown cause (idiopathic inflammatory bowel disease). In such cases, the animal is unable to ingest regular food because of inflammation and/or ulceration of the gut, leading to diarrhea. Inflammatory bowel disease in animals, particularly dogs and cats, is a condition whereby the intestinal tract is invaded by inflammatory cells such as lymphocytes, plasmacytes, eosinophils, and neutrophils. Other symptoms of IBD in animals may include vomiting, lack of appetite and weight loss.

Inflammation is a complex reaction of the immune system that involves the accumulation and activation of leucocytes and plasma proteins at sites of infection, toxin exposure or cell injury. Although inflammation serves as a protective function in controlling infections and promoting tissue repair, it can also, in case of dysregulation, cause tissue damage and disease. Gastrointestinal diseases such as inflammatory bowel disease (e.g. Crohn's disease, ulcerative colitis, and pouchitis), food allergies and atopic dermatitis resulting from food allergies are typically accompanied by aberrant intestinal inflammatory responses at different levels. The alleviation of this intestinal inflammation by balancing pro- and anti-inflammatory cytokines or induction of regulatory cytokines has been suggested as a possible treatment for these chronic diseases. There are a number of such cytokines, of which IFN-γ, IL-1, IL-6, IL-8, IL-12 and TNF-α, for example, are regarded as pro-inflammatory. For example, the role of TNF-α in gastrointestinal inflammation is known (Neurath, M. F.—Nature Reviews Immunology, (2014), 14, 329-342). Research efforts have focused on the development of anti-TNF-α agents to treat inflammatory gastrointestinal diseases such as Crohn's disease.

Some cytokines, such as IL-10 and TGF-β are regarded as anti-inflammatory. For example, IL-10 is known to suppress pro-inflammatory cytokine production by antigen-presenting cells and T cells. The involvement of IL-10 has been suggested in intestinal inflammation, and IL-10 deficiency has been associated with conditions such as inflammatory bowel disease [Leach, M. W., et al, Toxicol. Pathol. (1999), 27(1), 123-133].

The role of pro-inflammatory cytokines, such as IL-12 in the pathogenesis of inflammatory gastrointestinal conditions, particularly in inflammatory bowel disease is known, and IL-12 antagonists have been proposed as therapeutic agents for treating such conditions (Schmidt, C., et al., Pathobiology (2002-2003), 70(3), 177-183).

Serine protease inhibitors (serpins) are a superfamily of proteins found in eukaryotes (Gettins, 2002, Chemical Reviews, 102(12), 4751-4804) and prokaryotes (Kantyka et al., Biochimie, 92(11), 1644-1656). Serpins have been reported to be involved in a wide range of physiological processes, and have been implicated to play a role in controlling the proteases involved in intestinal inflammation. For example, Ivanov, D., et al. (J. Biol. Chem., (2006), 281 (25), 17246-17252) characterised and studied a B. Longum serpin which was found to inhibit human neutrophil elastase (HNE), and suggested a possible beneficial role for serpin inhibitors in intestinal inflammation [Vergnolle, N. —Gut (2016), 65(7), 1215-1224].

Other biomarkers in inflammatory gastrointestinal diseases include C-reactive protein (CRP) and other antibodies, 5-regulatory T-cells [Norouzinia, M., et al., Gastroenterology and Hepatology from Bed to Bench (2017), 10(3), 155-167].

Macrophages are tissue-based phagocytic cells derived from monocytes which play an important role in the innate immune response. They are activated by microbial components and, once activated, can themselves secrete both pro- and anti-inflammatory cytokines. He, F., et al (“Stimulation of the Secretion of Pro-Inflammatory Cytokines by Bifidobacterium Strains”—Microbiol. Immunol. (2002), 46(11), 781-785) investigated the ability of different bifidobacteria strains to affect the production of macrophage derived cytokines. They discovered that “adult type” bifidobacteria such as Bifidobacterium adolescentis and Bifidobacterium longum induced significantly more pro-inflammatory cytokine secretion than did “infant type” bifidobacteria such as Bifidobacterium bifidum, Bifidobacterium breve and Bifidobacterium infantis. In addition it was noted that B. adolescentis in particular did not stimulate production of the anti-inflammatory cytokine IL-10. They concluded that adult-type bifidobacteria may be more potent to amplify, but less able to down-regulate, the inflammatory response. However, more recently, attempts to identify the most promising anti-inflammatory probiotic strains for therapeutic use have indicated that taxonomic classification of a probiotic is not generally a reliable predictor of, e.g. the anti-inflammatory properties, of a particular probiotic strain.

Changes in intestinal barrier integrity and/or function are known to contribute to disease pathogenesis of inflammatory-gastrointestinal disorders, including IBD and IBS (Odenwald M. The intestinal epithelial barrier: a therapeutic target? Nature Reviews Gastroenterology & Hepatology, (2017), (14), 9-21). As such, alterations in gut barrier integrity/function have multiple consequences facilitating the onset of numerous diseases depending on other hits and on genetic and epigenetic constellations. Given the importance of the intestinal barrier for maintaining immune homeostasis and health, reinforcing the gastrointestinal intestinal barrier and/or improving gut barrier function represents a valuable new target for disease prevention and/or therapy. Probiotics represent one nutritional attempt to improve/reinforce intestinal barrier integrity and/or function (Ewaschuk J B et al., Secreted bioactive factors from Bifidobacterium infantis enhance epithelial cell barrier function, Am J Physiol Gastrointest Liver Physiol. 2008 November; 295(5):G1025-34).

In addition to altered intestinal barrier function, changes in microbiota composition and/or metabolic activity have also been associated with development of diseases including gastrointestinal disorders such as IBD. Series of nutritional attempts, including with probiotics, to restore microbiota have been considered as valuable approaches to reduce inflammation and improve disease condition in both humans and companion animals [Marchesi, J. R. et al. —Gut (2016), 65, 330-339; Harris, K. G and Chang, E. B.—Clin. Sci. (2018), 132 (18), 2013-2028; Parker, E. A., et al.—Nutrition (2018), 45, 125-134].

Therefore, an ongoing need exists to identify further therapies for treating or preventing inflammatory-related gastrointestinal disorders including inflammatory bowel disease.

### SUMMARY OF THE INVENTION

The present inventors have surprisingly discovered that a combination of two probiotic strains comprising a Bifidobacterium longum and a Bifidobacterium lactis, specifically Bifidobacterium longum ATCC CNCM I-2618, and Bifidobacterium lactis CNCM I-3446, optionally with Bifidobacterium longum ATCC BAA-999, can affect an increase in production or expression of anti-inflammatory cytokines, such as IL-10, and can decrease or suppress the production or expression of pro-inflammatory cytokines, such as IL-12. Moreover, a combination of Bifidobacterium longum and Bifidobacterium lactis, specifically Bifidobacterium longum ATCC CNCM I-2618 and Bifidobacterium lactis CNCM I-3446, optionally with Bifidobacterium longum ATCC BAA-999 has surprising been found to have effect on preventing inflammation-induced intestinal barrier dysfunction/permeability.

The probiotic combinations of the present invention are therefore useful for providing an effective therapy for the prevention or treatment of inflammation-related gastrointestinal disorders.

The probiotic combinations of the present invention may be employed in compositions for treating or preventing inflammation-related gastrointestinal disorders such as inflammatory bowel disease.

The invention further provides a method of treating inflammation-related gastrointestinal disorders such as an inflammatory bowel disorder, comprising administering a probiotic combination as described herein, to an individual suffering from, or susceptible to such a disorder.

### DESCRIPTION OF THE FIGURES

FIG. 1: Cytokine (IL-10) production in peripheral blood mononuclear cells stimulated with different probiotic strains and combinations

FIG. 2: Cytokine (IL-12p70) production in peripheral blood mononuclear cells stimulated with different probiotic strains and combinations

FIG. 3: Transepithelial electrical resistance (TEER) over TNFα/IFNγ treated controls to quantify inflammation-induced changes in barrier permeability with different probiotic strains and combinations thereof.

### DETAILED DESCRIPTION OF THE INVENTION

The following terms and definitions are used herein:

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

“Infant” refers to a child under the age of 12 months.

“Infant formula” refers to foodstuff intended for the complete nutrition of infants in the context of absence of breast-feeding during the first four to six months of life and as a complement to other foodstuffs up to the age of 12 months.

“Probiotic” refers to a microbial cell preparation or components of microbial cells with a beneficial effect on the health or well-being of the host (Salminen S, Ouwehand A. Benno Y. et al “Probiotics: how should they be defined”—Trend Food Sci. Technol. (1999), 10, 107-110).

A “child” refers to a person above the age of 12 months, but below the age of 10 years.

“Adolescent” refers to a person between the ages of 10-19 (based on the World Health Organisation (WHO) definition).

An “adult” refers to a person aged 20 or more.

A “puppy” refers to a dog that is less than 12 months old.

A “kitten” refers to a cat that is less than 12 months old.

“Cfu” refers to colony forming units, and is measured on a dry weight basis, unless otherwise indicated.

The term Bifidobacterium longum (B. longum) CNCM I-2618 is used interchangeably with Bifidobacterium longum (B. longum) NCC2705.

The term Bifidobacterium lactis (B. lactis) CNCM I-3446 is used interchangeably with Bifidobacterium lactis (B. lactis) NCC2818.

The term Bifidobacterium longum (B. longum) ATCC BAA-999 is used interchangeably with Bifidobacterium longum (B. longum) NCC3001.

The terms “improved intestinal barrier”, “Improved gut barrier”, “reinforcement of intestinal barrier” or “reinforcement of gut barrier” may encompass one or several of the following:
Improved barrier repair, such as (but not limited to) recovery of the integrity of the gastrointestinal barrier, such as repair of a disrupted barrier, reduction of permeability upon inflammatory challenge of the gastrointestinal mucosa, and mucosal repair.
Improved barrier maturation, such as (but not limited to) maturation and/or development of the barrier of an infant, child, adolescent, adult, dog, puppy, cat or kitten.
Improved barrier structure, such as (but not limited to) strengthening of the gastrointestinal barrier, integrity of the gastrointestinal barrier, tight junction structure, and intestinal epithelial lining integrity.
Improved barrier function, such as improvement of gastrointestinal barrier resistance, reduction of gastrointestinal barrier permeability, such as a reduction in translocation of pathogens from luminal sites to the mucosa, such as a reduction in translocation of commensal bacteria from luminal sites to the mucosa, such as a reduction in penetration of allergens from luminal sites to the mucosa, such as a reduction in transfer of toxic compounds from luminal sites to the mucosa, and reduction of disease susceptibility.
Improved barrier protection, such as (but not limited to) prevention of barrier dysfunction, prevention of barrier leakiness, protection of tight junction structure, protection of the intestinal epithelial lining integrity.

The terms B. longum CNCM I-2618, B. lactis CNCM I-3446 and B. longum ATCC BAA-999 are intended to include the bacterium, parts of the bacterium and/or a growth medium fermented by the bacterium.

The B. longum CNCM I-2618, B. lactis CNCM I-3446 and B. longum ATCC BAA-999, may each be used as living bacterium as well as inactivated non-replicating bacterial species. “Non-replicating” means that no viable cells and/or colony forming units can be detected by classical plating methods. Such classical plating methods are summarized in the microbiology book: James Monroe Jay, Martin J. Loessner, David A. Golden. 2005. Modern food microbiology. 7th edition, Springer Science, New York, N. Y. 790 p. Typically, the absence of viable cells can be shown as follows: no visible colony on agar plates or no turbidity in liquid growth medium after inoculation with different concentrations of bacterial preparations (“non replicating' samples”) and incubation under appropriate conditions (aerobic and/or anaerobic atmosphere for at least 24 h).

It is preferred that at least part of the B. longum CNCM I-2618, B. lactis CNCM I-3446 and (when present) B. longum ATCC BAA-999, are alive in the combination or composition and preferably arrive alive in the intestine. This way they can persist in the intestine, be metabolically active and may increase their effectiveness. They may also be effective by interacting with the commensal bacteria and/or the host. For special sterile food products or medicaments, for example, it might be preferable that B. longum CNCM I-2618, B. lactis CNCM I-3446 and (when present) B. longum ATCC BAA-999 are present in a non-replicating form in the combination or composition. Hence, in one embodiment of the present invention at least a part of the B. longum CNCM I-2618, B. lactis CNCM I-3446 and (when present) B. longum ATCC BAA-999, are non-replicating in the combination or composition.

In one embodiment, the present invention provides a probiotic combination for use in the reduction of gastrointestinal inflammation or for treatment or prevention of inflammatory-related gastrointestinal disorders, wherein the probiotic combination comprises Bifidobacterium longum CNCM I-2618 and Bifidobacterium lactis CNCM I-3446. Optionally, the probiotic combination can further comprise Bifidobacterium longum ATCC BAA-999.

In another embodiment the present invention provides a probiotic combination comprising B. longum CNCM I-2618 and B. lactis CNCM I-3446, and optionally further comprising B. longum ATCC BAA-999, for decreasing or suppressing the production or expression of a pro-inflammatory cytokine (preferably IL-12) and/or increasing the production or expression of an anti-inflammatory cytokine (preferably IL-10) or for regulating the serum concentration ratios of IL-10 and IL-12.

In another embodiment, the present invention provides a probiotic combination comprising B. longum CNCM I-2618 and B. lactis CNCM I-3446, and optionally further comprising B. longum ATCC BAA-999, for modulating T regulatory cells or Th17/Treg differentiation; or for modulating a quorum sensing signaling inhibitor.

In another embodiment, the present invention provides a probiotic combination comprising B. longum CNCM I-2618 and B. lactis CNCM I-3446, and optionally further comprising B. longum ATCC BAA-999, for increasing the population of beneficial bacteria, such as bifidobacteria and lactobacilli, in the gut.

In another embodiment, the present invention provides a probiotic combination comprising B. longum CNCM I-2618 and B. lactis CNCM I-3446, and optionally further comprising B. longum ATCC BAA-999, for promoting the production or expression of defensins or mucins; for reducing oxidative stress or inflammatory markers such as CRP.

In another embodiment, the present invention provides a probiotic combination comprising B. longum CNCM I-2618 and B. lactis CNCM I-3446, and optionally further comprising B. longum ATCC BAA-999, for promoting microbiota metabolic function, including but not limited to, short-chain fatty acids such as butyrate.

In another embodiment, the present invention provides a probiotic combination comprising B. longum CNCM I-2618 and B. lactis CNCM I-3446, and optionally further comprising B. longum ATCC BAA-999, for supporting or promoting mucosal tissue healing.

In another embodiment, the present invention provides a probiotic combination comprising B. longum CNCM I-2618 and B. lactis CNCM I-3446, and optionally further comprising B. longum ATCC BAA-999, for reinforcing or improving the intestinal barrier.

In one embodiment, the present invention provides a probiotic combination comprising B. longum CNCM I-2618 and B. lactis CNCM I-3446, and optionally further comprising B. longum ATCC BAA-999, for improving intestinal barrier repair.

Improved intestinal barrier permeability results in improved barrier repair, which leads to improved or reinforced intestinal barrier. Thus, in an embodiment, said improvement to the intestinal barrier is improved barrier permeability (such as reduction in intestinal barrier permeability).

In one embodiment, the present invention provides a probiotic combination comprising B. longum CNCM I-2618 and B. lactis CNCM I-3446, and optionally further comprising B. longum ATCC BAA-999, for improving intestinal barrier function.

Improved intestinal barrier permeability results in improved barrier function, which leads to improved or reinforced intestinal barrier. Thus, in an embodiment, said improvement to the intestinal barrier is improved barrier permeability (such as reduction in intestinal barrier permeability).

In one embodiment, improved intestinal barrier results in reduction in pathogens, allergens and/or toxic compounds migrating from the gut into the body through the intestinal barrier.

The probiotic combination comprising B. longum CNCM I-2618 and B. lactis CNCM I-3446, and optionally further comprising B. longum ATCC BAA-999 is preferably for use in the treatment or prevention of inflammatory-related gastrointestinal disorders. The inflammatory-related gastrointestinal disorder is preferably one which is modulated by pro-inflammatory cytokines (preferably IL-12) and/or anti-inflammatory cytokines (preferably IL-10). Preferably, the inflammatory-related gastrointestinal disorder is one which is modulated by IL-10 and/or IL-12; or wherein the inflammatory-related gastrointestinal disorder is modulated by T regulatory cells or Th17/Treg differentiation; or wherein the inflammatory-related gastrointestinal disorder is modulated by a quorum sensing signaling inhibitor.

In any aspect or embodiment of the present invention, the inflammatory-related gastrointestinal disorder is modulated by an imbalance of beneficial bacteria, such as bifidobacteria and lactobacilli; or wherein the inflammatory-related gastrointestinal disorder is modulated by defensins or mucins, or wherein the inflammatory-related gastrointestinal disorder is modulated by oxidative stress or inflammatory markers such as CRP.

The present invention further provides a probiotic combination comprising B. longum CNCM I-2618 and B. lactis CNCM I-3446, and optionally further comprising B. longum ATCC BAA-999, for the treatment or prevention of inflammatory bowel disease, particularly wherein the inflammatory bowel disease is selected from the group consisting of: colitis, ulcerative colitis, chronic enteropathy, Crohn's disease and pouchitis; or wherein the inflammatory-related gastrointestinal disorder is food responsive diarrheal disease.

Preferably, the probiotic combination of any aspect or embodiment of the present invention is for use in the treatment or prevention of ulcerative colitis, Crohn's disease or food responsive diarrhea.

Preferably, the treatment or prevention of an inflammatory-related gastrointestinal disorder using the probiotic combination of the invention comprises increasing the production or expression of an anti-inflammatory cytokine (preferably IL-10), and/or comprises decreasing or suppressing the production or expression of a pro-inflammatory cytokine (preferably IL-12). More preferably, the treatment or prevention of an inflammatory-related gastrointestinal disorder using the probiotic combination of the present invention comprises regulating the serum concentration ratios of IL-10 and IL-12.

The subject to be treated is preferably a mammal, preferably a human or a companion animal (pet), preferably wherein the subject is a child, an infant, an adolescent or an adult human, a dog, a puppy, a cat or a kitten.

Thus, the present invention further provides a probiotic combination comprising B. longum CNCM I-2618 and B. lactis CNCM I-3446, and optionally further comprising B. longum ATCC BAA-999 for treatment of feline or canine inflammatory bowel disease, preferably feline or canine food-responsive diarrheal disease or idiopathic inflammatory bowel disease. The probiotic combination is preferably in the form of a composition, more preferably a pet food (particularly a dry pet food) or a pet nutritional supplement or a veterinary composition (particularly a tablet, a capsule or a dry powder).

In another embodiment, the subject to be treated is a dog, puppy, cat or kitten. Thus, the present invention further provides a probiotic combination comprising B. longum CNCM I-2618 and B. lactis CNCM I-3446, and optionally further comprising B. longum ATCC BAA-999 for treatment of feline or canine inflammatory bowel disease, preferably feline or canine food-responsive diarrheal disease or idiopathic inflammatory bowel disease. The probiotic combination is preferably in the form of a composition, more preferably a pet food, a pet nutritional supplement or a veterinary composition.

In yet another embodiment, the subject to be treated in a child, an infant, an adolescent or an adult human. Thus, the present invention further provides a probiotic combination comprising B. longum CNCM I-2618 and B. lactis CNCM I-3446, and optionally further comprising B. longum ATCC BAA-999 for the treatment of inflammatory bowel disease in these subjects. The probiotic combination is preferably in the form of a composition, more preferably a food, a nutritional supplement or a pharmaceutical composition (particularly a tablet, a capsule, granules, or a dry powder).

In any embodiment of the present invention, the probiotic combination contains B. longum CNCM I-2618 and B. lactis CNCM I-3446 as the only probiotic bacteria. The probiotic combination is preferably in the form of a composition, more preferably a food, a nutritional supplement or a pharmaceutical or veterinary composition.

In any embodiment of the present invention, the probiotic combination comprises B. longum CNCM I-2618, B. lactis CNCM I-3446 and B. longum ATCC BAA-999. Alternatively, the probiotic combination according to any embodiment of the present invention may comprise B. longum CNCM I-2618, B. lactis CNCM I-3446 and B. longum ATCC BAA-999 as the only probiotic bacteria. The probiotic combination is preferably in the form of a composition, more preferably a food, a nutritional supplement or a pharmaceutical or veterinary composition.

The probiotic combination may be in the form of a composition as described in any embodiment, wherein the composition contains B. longum CNCM I-2618 and B. lactis CNCM I-3446 as the only probiotic bacteria. The probiotic composition is preferably in the form of a food, a nutritional supplement or a pharmaceutical or veterinary composition.

The probiotic combination may be in the form of a composition as described in any embodiment, wherein the composition comprises B. longum CNCM I-2618, B. lactis CNCM I-3446 and B. longum ATCC BAA-999. Alternatively, the probiotic combination may be in the form of a composition as described in any embodiment, wherein the composition contains B. longum CNCM I-2618, B. lactis CNCM I-3446 and B. longum ATCC BAA-999 as the only probiotic bacteria. The probiotic composition is preferably in the form of a food, a nutritional supplement or a pharmaceutical or veterinary composition.

Any suitable dose of the probiotic combination may be used. Preferably, in any embodiment of the invention, the probiotic combination comprises B. longum CNCM I-2618 and B. lactis CNCM I-3446, and optionally further comprising B. longum ATCC BAA-999, wherein each probiotic is administered to a subject in an amount equating to 10⁸ to 10¹² cfu per day.

Although the probiotic components of the combination can be used without further processing, the probiotic combination according to any embodiment of the invention is preferably administered in the form of a composition. Suitable compositions comprise B. longum CNCM I-2618 and B. lactis CNCM I-3446, and optionally further comprising B. longum ATCC BAA-999 in the form of a pharmaceutical or veterinary formulation comprising one or more pharmaceutically or veterinary acceptable excipients, a nutritional formulation (e.g. including a nutritional supplement), a tube-feed formulation, a dietary supplement, a functional food, a beverage product and a pet care product (e.g. a pet food, or a pet nutritional supplement).

The pharmaceutical or veterinary formulation is preferably/may be in the form of a tablet, a capsule, granules, or a powder.

According to any embodiment of the present invention, the composition may comprise an amount equating to 10⁸ to 10¹² cfu per day, either as a single dose, or as multiple doses.

Also provided is a probiotic combination comprising B. longum CNCM I-2618 and B. lactis CNCM I-3446, and optionally further comprising B. longum ATCC BAA-999, for use in the manufacture of a composition for use in the reduction of gastrointestinal inflammation, or for the treatment or prevention of inflammatory-related gastrointestinal disorders.

The invention further provides a method for reducing gastrointestinal inflammation, or for treating or preventing inflammatory bowel disease in a subject comprising the step of administering to said subject a probiotic combination, wherein the probiotic combination comprises B. longum CNCM I-2618 and B. lactis CNCM I-3446, and optionally further comprises B. longum ATCC BAA-999.

The probiotic combination of the present invention comprising B. longum CNCM I-2618 and B. lactis CNCM I-3446, and optionally further comprising B. longum ATCC BAA-999, may be provided for simultaneous or sequential administration of each of the probiotics. Alternatively, the probiotic combination may be formulation as a single composition.

The probiotic combination comprising Bifidobacterium longum CNCM I-2618 and Bifidobacterium lactis CNCM I-2446 and optionally further comprising B. longum ATCC BAA-999, may be administered as a composition (e.g. a capsule, a tablet, granules or a powder) containing, for example, 10$-10¹² colony forming units (cfu) of each probiotic component, or may be incorporated in a nutritional composition such as a nutritionally complete formula (for example an infant formula or a clinical nutrition product), a dairy product, a beverage powder, a dehydrated soup, a dietary supplement, a meal replacement, a nutritional bar, a cereal, a confectionery product or a dry pet food.

In one embodiment, the combination may be in the form of a single capsule comprising both B. longum CNCM I-2618 and B. lactis CNCM I-2446, or a single capsule comprising B. longum CNCM I-2618, B. lactis CNCM I-2446 and B. longum ATCC BAA-999.

Alternatively, the combination may be provided as separate capsules, comprising B. longum CNCM I-2618 in one capsule and B. lactis CNCM I-2446 in another capsule, for simultaneous or sequential administration; or the combination may be provided as separate capsules comprising B. longum CNCM I-2618, B. lactis CNCM I-2446 and B. longum ATCC BAA-999 as separate capsules for simultaneous or sequential administration.

When incorporated in a nutritional composition, B. longum CNCM I-2618 and B. lactis CNCM I-3446 and optionally B. longum ATCC BAA-999 may each be present in the composition in an amount equivalent to between 10⁴ and 10¹² cfu/g (dry weight). These expressions of quantity include the possibilities that the bacteria are live, inactivated or dead or even present as fragments such as DNA or cell wall materials or as metabolites. In other words, the quantities of bacteria are expressed in terms of the colony forming ability of that quantity of bacteria as if all the bacteria were live irrespective of whether they are, in fact, live, inactivated or dead, fragmented or a mixture of any or all of these states. Preferably each of the B. longum CNCM I-2618, B. lactis CNCM I-3446 and B. longum ATCC BAA-999 (when present) is present in an amount equivalent to between 10⁵ to 10¹⁰, more preferably 10⁷ to 10¹⁰ cfu/g of dry composition.

In embodiments of the present invention wherein the probiotic combination further comprises B. Longum ATCC BAA-999, the B. Longum ATCC BAA-999 may be present either in the same composition or in a separate composition for simultaneous or sequential administration. For example, in the above described capsules, the B. Longum ATCC BAA-999 may be enclosed in capsules with the B. longum CNCM I-2618 and B. lactis CNCM I-3446, wherein each capsule contains 10$-10¹² colony forming units (cfu). Likewise, the composition comprising B. longum CNCM I-2618, B. lactis CNCM I-3446 and B. Longum ATCC BAA-999 may be incorporated in a nutritional composition such as a nutritionally complete formula (for example an infant formula or a clinical nutrition product), a dairy product, a beverage powder, a dehydrated soup, a dietary supplement, a meal replacement, a nutritional bar, a cereal, a confectionery product or a dry pet food.

B. longum CNCM I-2618 and B. lactis CNCM I-3446 may each be cultured according to any suitable method and prepared for encapsulation or addition to a nutritional composition by freeze-drying or spray-drying for example. Alternatively, they may be purchased already prepared in a suitable form for addition to food products.

ATCC BAA-999 is commercially available and may be obtained from Morinaga Milk Industry Co. Ltd. of Japan under the trade mark BB536. It may be cultured according to any suitable method and prepared for encapsulation or addition to a nutritional composition by freeze-drying or spray-drying for example. Alternatively, it may be purchased already prepared in a suitable form for addition to food products.

A nutritionally complete formula for use in the present invention may comprise a source of protein, preferably a dietary protein such as an animal protein (for example milk, meat or egg protein), a vegetable protein (for example soy, wheat, rice or pea protein); mixtures of free amino acids; or combinations thereof. Milk proteins such as casein and whey protein and soy proteins are particularly preferred. The composition may also contain a source of carbohydrates and a source of fat.

If the formula includes a fat source, it preferably provides 5% to 55% of the energy of the formula; for example 20% to 50% of the energy. The lipids making up the fat source may be any suitable fat or fat mixture. Vegetable fats such as soy oil, palm oil, coconut oil, safflower oil, sunflower oil, corn oil, canola oil, and lecithins are particularly suitable. Animal fats such as milk fat may also be added if desired.

If the formula includes a carbohydrate source, it preferably provides 40% to 80% of the energy of the formula. Any suitable carbohydrate may be used, for example sucrose, lactose, glucose, fructose, corn syrup solids, maltodextrins, and mixtures thereof. Dietary fibre may also be added if desired. The dietary fibre may be from any suitable origin, including for example soy, pea, oat, pectin, guar gum, gum Arabic, fructo-oligosaccharides, galacto-oligosaccharides, sialyl-lactose and oligosaccharides derived from animal milks. Suitable vitamins and minerals may be included in the nutritional formula in an amount to meet the appropriate guidelines.

The compositions of the present invention may further include a prebiotic. Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such as fructo-oligosaccharides (FOS), inulin, xylo-oligosaccharides (XOS), polydextrose or any mixture thereof. In a particular embodiment, the prebiotics may be fructo-oligosaccharides and/or inulin. An example is a combination of 70% short chain fructo-oligosaccharides and 30% inulin, which is registered by Nestle under the trademark “Prebio 1”.

One or more food grade emulsifiers may be incorporated into the nutritional formula if desired; for example diacetyl tartaric acid esters of mono- and di-glycerides, lecithin and mono- and di-glycerides. Similarly suitable salts and stabilisers may be included.

The nutritionally complete formula may be prepared in any suitable manner. For example, the protein source, the carbohydrate source, and the fat source may be blended together in appropriate proportions. If used, the emulsifiers may be included in the blend. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to a temperature in the range from about 60° C. to about 85° C.; for example by flash cooling. The liquid mixture may then be homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture is conveniently standardised at this point.

The homogenised mixture may then be transferred to a suitable drying apparatus such as a spray drier or freeze drier and converted to powder. The powder should have a moisture content of less than about 5% by weight. The B. longum CNCM I-2618, and/or B. lactis CNCM I-3446, and/or B. Longum ATCC BAA-999 may be added to the powder in the desired quantity by dry mixing.

A dry pet food for use in the present invention may include any one or more of a carbohydrate source, a protein source and lipid source.

Any suitable carbohydrate source may be used. Preferably, the carbohydrate source is provided in the form of grains, flours or starches. For example, the carbohydrate source may be rice, barley, sorghum, millet, oat, corn meal or wheat flour. Simple sugars such as sucrose, glucose and corn syrups may also be used. The amount of carbohydrate provided by the carbohydrate source may be selected as desired. For example, the pet food may contain up to about 60% by weight of carbohydrate.

Suitable protein sources may be selected from any suitable animal or vegetable protein source; for example muscular or skeletal meat, meat and bone meal, poultry meal, fish meal, milk proteins, corn gluten, wheat gluten, soy flour, soy protein concentrates, soy protein isolates, egg proteins, whey, casein, gluten, and the like. For elderly animals, it is preferred for the protein source to contain a high quality animal protein. The amount of protein provided by the protein source may be selected as desired. For example, the pet food may contain about 12% to about 70% by weight of protein on a dry basis.

The pet food may contain a fat source. Any suitable fat source may be used.

Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used. In addition to essential fatty acids (linoleic and alpha-linoleic acid) the fat source may include long chain fatty acids. Suitable long chain fatty acids include gamma linoleic acid, stearidonic acid, arachidonic acid, eicosapentanoic acid, and docosahexanoic acid. Fish oils are a suitable source of eicosapentanoic acids and docosahexanoic acid. Borage oil, blackcurrant seed oil and evening primrose oil are suitable sources of gamma linoleic acid. Rapeseed oil, soybean oil, linseed oil and walnut oil are suitable sources of alpha-linoleic acid. Safflower oils, sunflower oils, corn oils and soybean oils are suitable sources of linoleic acid. Olive oil, rapeseed oil (canola), high oleic sunflower oil, safflower oil, peanut oil, and rice bran oil are suitable sources of monounsaturated fatty acids. The amount of fat provided by the fat source may be selected as desired. For example, the pet food may contain about 5% to about 40% by weight of fat on a dry basis. Preferably, the pet food has a relatively reduced amount of fat.

The choice of the carbohydrate, protein and lipid sources is not critical and will be selected based upon nutritional needs of the animal, palatability considerations, and the type of product produced. Further, various other ingredients, for example, sugar, salt, spices, seasonings, vitamins, minerals, flavouring agents, gums, and probiotic microorganisms may also be incorporated into the pet food as desired.

For elderly pets, the pet food preferably contains proportionally less fat than pet foods for younger pets. Further, the starch sources may include one or more of oat, rice, barley, wheat and corn.

The pet food may be produced by extrusion cooking, although baking and other suitable processes may be used. When extrusion cooked, the pet food is usually provided in the form of a kibble. The probiotic components may preferably be coated onto or filled into the dried pet food. A suitable process is described in European Patent Application No 0862863.

The probiotic combination of present invention, and compositions thereof may be used to treat or manage chronic or acute intestinal inflammation caused by a disease of the gastrointestinal tract such as inflammatory bowel disease or colitis, post-infective inflammation or chronic sub-clinical inflammation in the elderly as well as in circumstances where it is desired to prevent inflammation in the sense of prophylaxis in individuals susceptible to such disorders.

Typically, the composition may be selected from the group consisting of a food composition, a pet food composition, a dietary supplement, a nutraceutical, a nutritional formula, a drink, and/or a medical composition.

Examples of food compositions that are applicable to the present invention are yoghurts, milk, flavoured milk, ice cream, ready to eat desserts, powders for re-constitution with, e.g., milk or water, chocolate milk drinks, malt drinks, ready-to-eat dishes, instant dishes or drinks for humans or food compositions representing a complete or a partial diet intended for pets or livestock. Consequently, in one embodiment the composition according to the present invention is a food product intended for humans, pets or livestock, and preferably humans and pets. In a preferred embodiment, the composition is a food product or a dietary supplement intended for humans (infant, child, adolescent, or adult) or companion animals (pets) (preferably dog, puppy, cat or kitten).

The composition of the present invention may further contain protective hydrocolloids (such as gums, proteins, modified starches), binders, film forming agents, encapsulating agent(s)/material(s), wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, lignin sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

The composition may be a nutritionally complete formula. The composition according to the invention may comprise a source of protein.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof.

The proteins may be intact or hydrolysed or a mixture of intact and hydrolysed proteins. It may be desirable to supply partially hydrolysed proteins (degree of hydrolysis between 2 and 20%), for example for human subjects and/or animals at risk of developing cows' milk allergy.

Furthermore, pre-hydrolysed protein sources are generally easier digested and absorbed by an impaired gastro-intestinal tract.

If hydrolysed proteins are required, the hydrolysis process may be carried out as desired and as is known in the art. It may be desirable to supply partially hydrolysed proteins (degree of hydrolysis between 2 and 20%).

For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

The composition may also contain a source of carbohydrates and a source of fat. If the composition includes a fat source, the fat source preferably provides 5% to 40% of the energy of the composition; for example 20% to 30% of the energy. A suitable fat profile may be obtained using a blend of canola oil, corn oil and high-oleic acid sunflower oil.

A source of carbohydrate may be added to the composition.

The source of carbohydrates preferably provides 40% to 80% of the energy of the composition. Any suitable carbohydrate may be used, for example sucrose, lactose, glucose, fructose, corn syrup solids, maltodextrins, and mixtures thereof. Dietary fibre may also be added if desired. Dietary fibre passes through the small intestine undigested by enzymes and functions as a natural bulking agent and laxative. Dietary fibre may be soluble or insoluble and in general a blend of the two types is preferred. Suitable sources of dietary fibre include soy, pea, oat, pectin, guar gum, partially hydrolysed guar gum, gum Arabic, fructo-oligosaccharides, acidic oligosaccharides, galacto-oligosaccharides, sialyl-lactose and oligosaccharides derived from animal milks. A preferred fibre blend is a mixture of inulin with shorter chain fructo-oligosaccharides. Preferably, if fibre is present, the fibre content is between 2 and 40 g/l of the composition as consumed, more preferably between 4 and 10 g/l.

The composition may also contain minerals and micronutrients such as trace elements and vitamins in accordance with the recommendations of Government bodies such as the USRDA. For example, the composition may contain per daily dose one or more of the following micronutrients in the ranges given: 300 to 500 mg calcium, 50 to 100 mg magnesium, 150 to 250 mg phosphorus, 5 to 20 mg iron, 1 to 7 mg zinc, 0.1 to 0.3 mg copper, 50 to 200 μg iodine, 5 to 15 μg selenium, 1000 to 3000 μg beta carotene, 10 to 80 mg Vitamin C, 1 to 2 mg Vitamin B1, 0.5 to 1.5 mg Vitamin B6, 0.5 to 2 mg Vitamin B2, 5 to 18 mg niacin, 0.5 to 2.0 μg Vitamin B12, 100 to 800 μg folic acid, 30 to 70 μg biotin, 1 to 5 μg Vitamin D, 3 to 10 μg Vitamin E.

One or more food grade emulsifiers may be incorporated into the composition if desired; for example diacetyl tartaric acid esters of mono- and di-glycerides, lecithin and mono- and di-glycerides. Similarly suitable salts and stabilisers may be included.

The composition may be orally and/or enterally administrable; for example in the form of a powder for re-constitution with milk or water.

The compositions are administered in an amount sufficient to at least partially treat or arrest the symptoms of the inflammation-related gastrointestinal disease and its complications. An amount adequate to accomplish this is defined as “a therapeutically effective dose”. Amounts effective for this purpose will depend on a number of factors known to those of skill in the art such as the severity of the disease and the weight and general state of the patient.

In prophylactic applications, compositions according to the invention are administered to a patient susceptible to or otherwise at risk of a particular disease in an amount that is sufficient to at least partially reduce the risk of developing a disease. Such an amount is defined to be “a prophylactic effective dose”. Again, the precise amounts depend on a number of patient specific factors such as the patient's state of health and weight.

Generally, B. longum CNCM I-2618, B. lactis CNCM I-3446 and (where present) B. Longum ATCC BAA-999, will each be administered in a therapeutically effective dose and/or in a prophylactic effective dose.

If B. longum CNCM I-2618, B. lactis CNCM I-3446 and (where present) B. Longum ATCC BAA-999 are present in a viable form, it is theoretically effective in any concentration considering the fact that these bacteria can colonize the gut and multiply. For the compositions of the present invention, it is generally preferred that a daily dose of the composition comprises between 10⁴ and 10¹² cfu of each of the probiotic agents. A particular suitable daily dose of each of the probiotics is from 10⁸ to 10¹² cfu.

In the case of inactivated and/or non-replicating B. longum CNCM I-2618, B. lactis CNCM I-3446, and (where present) B. Longum ATCC BAA-999, it is generally preferred that the composition of the present invention comprises between 10² and 10¹² non-replicating cells of Bifidobacterium longum ATCC BAA-999, per gram of the dry weight of the composition. A particular suitable dose of each of the probiotics, is from 10³ to 10¹² non-replicating cells, more preferably from 10⁵ to 10⁸ non-replicating cells per gram of the dry weight of the composition.

Obviously, non-replicating micro-organisms do not form colonies, consequently, the term cells is to be understood as the amount of non-replicating micro-organisms that is obtained from the specified amount of replicating bacterial cells. This includes micro-organisms that are inactivated, non-viable or dead or present as fragments such as DNA or cell wall materials.

The composition of the present invention may be provided in powder form having a water activity of lower than 0.2, for example in the range of 0.19-0.05, preferably smaller than 0.15.

The composition may be a shelf stable powder. The low water activity provides this shelf stability and ensures that probiotic microorganisms, will remain viable even after long storage times.

Water activity or a_{w} is a measurement of the energy status of the water in a system. It is defined as the vapour pressure of water divided by that of pure water at the same temperature; therefore, pure distilled water has a water activity of exactly one.

Additionally or alternatively, the probiotic microorganism B. longum CNCM I-2618, B. lactis CNCM I-3446 and (where present) B. Longum ATCC BAA-999 may be provided in an encapsulated form.

It has been found that encapsulation of the bacteria has therapeutic and technical advantages. Encapsulation increases the survival of the bacteria and thus the number of live bacteria which arrive in the intestine. Furthermore, the bacteria are gradually released allowing a prolonged action of the bacteria on the health of the subject. Bacteria may be micro-encapsulated, for example as described by FR2443247 (Societe des Produits Nestle), incorporated herein by reference. Briefly, the bacteria may be freeze or spray dried and incorporated into a gel.

The invention will now be further described by the reference to the following example.

### Example

### Preparation of Bacteria:

The day before the assay, three selected bacterial strains from the Nestle Culture Collection NCC 3001, 2818 and 2705 were cultured in 10 ml MRS+cysteine and grown for 16 hrs at 37° C. in anaerobic conditions.

Bacterial cultures were centrifuged at 5000 rpm 5 min (room temperature). Bacterial pellet were resuspended in cold phosphate buffered saline (PBS) (10 mL). The optical density of each bacterial culture was measured at 600 nm. Adjusted bacteria preparations in RPMI culture medium were set to have 5×10⁶ CFU/ml and 1×10⁷ CFU/ml according to pre-test of bacterial colony forming unit counting on selective agar medium that validated correspondence of OD and CFU.

### Preparation of Peripheral Blood Mononuclear Cell (PBMC):

PBMC isolated from three healthy donors were washed once in PBS. After a centrifugation at 500 g for 5 minutes, the cell pellet was resuspended in 2 ml RPMI+10% fetal calf serum (FCS). Cells were counted and preparations adapted to have 2×10⁶ cells/ml.

Stimulation of PBMC with Bacteria:

PBMC were seeded in 12 wells culture plate (500 μL) then bacteria preparations were added (500 μL). Co-cultures were incubated for 24 hrs at 37° C. with 10% CO₂.

For the cytokine analyses, the supernatants were centrifuged for 5 minutes at 500 g and transferred in a new tube. The samples were stored at −20° C. until assessment. Cytokines IL-10 and IL12 were measured by ELISA (IL-10 and IL-12 ELISA, R&D Systems, MN).

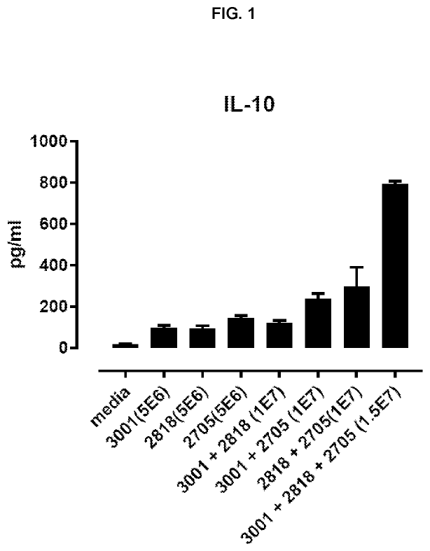

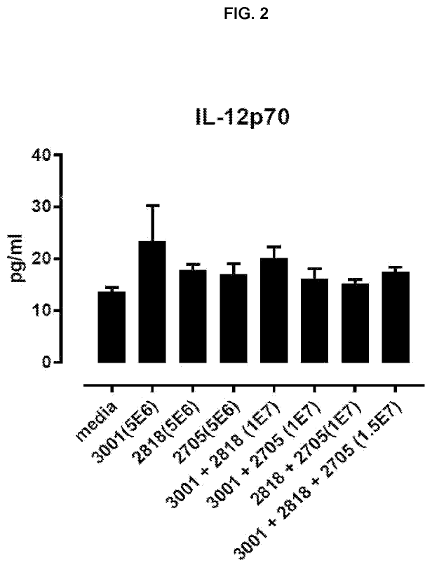

The results are shown in FIGS. 1 and 2.

### Assessment of Intestinal Barrier Permeability

Caco-2 and HT-29-MTX cells were co-cultured in 12 well cultures plates on polystyrene filter inserts at a ratio of 3:1. Upon differentiation (14 days), co-cultures were pre-incubated with bacteria preparations (5×10⁶) for 24 h prior to basolateral stimulus with TNFα (0.6 ng/mL) and IFNγ (2.5 ng/mL) to alter barrier integrity (control). Transepithelial electrical resistance (TEER) was measured after 16 h to quantify inflammation-induced changes in barrier permeability illustrated as percent increase in TEER over TNFα/IFNγ treated controls.

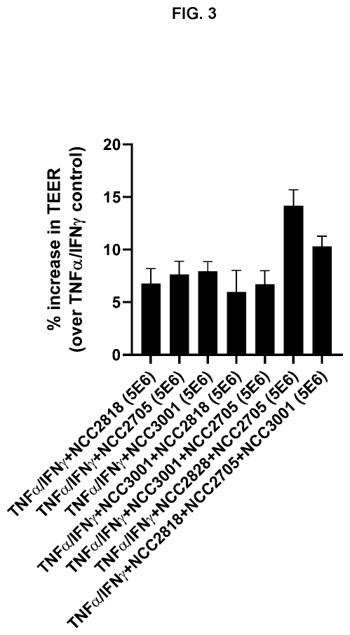

The results are shown in FIG. 3.

As clearly shown in the figures, a combination of B. longum CNCM I-2618 and B. lactis according to the present invention are surprisingly effective at increasing the production of the anti-inflammatory cytokine IL-10 (FIG. 1). The combination of B. longum CNCM I-2618, B. lactis CNCM I-3446 also surprisingly suppresses the production of IL-12 compared with the B. longum CNCM I-2618 and B. lactis CNCM I-3446 when employed separately (FIG. 2).

FIG. 1 further demonstrates a surprising synergistic effect on the increase in the production of the anti-inflammatory cytokine IL-10 when a triple combination of B. longum CNCM I-2618, B. lactis CNCM I-3446 and B. Longum ATCC BAA-999 of the invention is employed. The triple combination according to one aspect of the present invention further suppresses the production of IL-12 compared with B. longum CNCM I-2618, B. lactis CNCM I-3446 and B. Longum ATCC BAA-999 when employed separately (FIG. 2).

As clearly shown in FIG. 3 and according to the present invention, B. longum CNCM I-2618 and B. lactis CNCM I-3446 are surprisingly effective at preventing inflammation-induced barrier permeability compared to single strains or combinations of otherstrains. The triple combination of B. longum CNCM I-2618, B. lactis CNCM I-3446 and B. Longum ATCC BAA-999 according to one aspect of the present invention also shows prevention of inflammation-induced barrier disruption (FIG. 3).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A composition comprising a probiotic combination, wherein the probiotic combination comprises Bifidobacterium longum CNCM I-2618 and Bifidobacterium lactis CNCM I-3446.

14. A composition according to claim 13, wherein said probiotic combination further comprises Bifidobacterium longum ATCC BAA-999.

15. A composition according to claim 13, wherein the composition comprises each probiotic in the probiotic combination in an amount of 10³ to 10¹² cfu per gram of the dry weight of the probiotic composition.

16. A composition according to claim 13, wherein the composition is selected from the group consisting of: a pharmaceutical formulation, a veterinary formulation, a nutritional formulation, a tube-feed formulation, a dietary supplement, a functional food, a beverage product and a pet care product.

17. (canceled)

18. (canceled)

19. A method for reducing gastrointestinal inflammation, reinforcing intestinal barrier, or for treating or preventing inflammatory bowel disease in a subject comprising the step of administering to said subject a probiotic combination, wherein the probiotic combination is Bifidobacterium longum CNCM I-2618 and Bifidobacterium lactis CNCM I-3446.

20. A method according to claim 19, wherein the probiotic combination further comprises Bifidobacterium longum ATCC BAA-999.

21. A method for prevention of inflammation-induced intestinal barrier dysfunction, reduction of intestinal barrier permeability, improvement of barrier repair, improvement of barrier function and/or reinforcement/protection of intestinal barrier in a subject, comprising the step of administering to said subject a probiotic combination, wherein the probiotic combination comprises Bifidobacterium longum CNCM I-2618 and Bifidobacterium lactis CNCM I-3446.

22. (canceled)

23. The method of claim 21, wherein the probiotic combination further comprises Bifidobacterium longum ATCC BAA-999.

24. A method for reduction of gastrointestinal inflammation or for treatment or prevention of inflammatory-related gastrointestinal disorders in a subject, comprising the step of administering to said subject a probiotic combination, wherein the probiotic combination comprises Bifidobacterium longum CNCM I-2618 and Bifidobacterium lactis CNCM I-3446.

25. A method according to claim 24, wherein the probiotic combination further comprises Bifidobacterium longum ATCC BAA-999.

26. The method according to claim 24, wherein the inflammatory-related gastrointestinal disorder is modulated by a pro-inflammatory and/or anti-inflammatory cytokine; wherein the inflammatory-related gastrointestinal disorder is modulated by IL-10 and/or IL-12; wherein the inflammatory-related gastrointestinal disorder is modulated by T regulatory cells or Th17/Treg differentiation; or wherein the inflammatory-related gastrointestinal disorder is modulated by a quorum sensing signaling inhibitor.

27. The method according to claim 24, wherein the inflammatory-related gastrointestinal disorder is modulated by an imbalance of beneficial bacteria; wherein the inflammatory-related gastrointestinal disorder is modulated by defensins or mucins; or wherein the inflammatory-related gastrointestinal disorder is modulated by oxidative stress or inflammatory markers such as CRP.

28. The method according to claim 24, wherein the probiotic composition decreases or suppresses the production or expression of a pro-inflammatory cytokine; increases the production or expression of an anti-inflammatory cytokine and/or decreases or suppresses the production or expression of a pro-inflammatory cytokine; regulates the concentration ratios of IL-10 and IL-12; increases the population of beneficial bacteria in the gut; promotes the production or expression of defensins or mucins; reduces oxidative stress or inflammatory markers; promotes microbiota metabolic function; supports or promotes tissue healing or reinforces or improves gut barrier; improves intestinal barrier repair and/or function; improves or reinforces intestinal barrier permeability; modulates T regulatory cells or Th17/Treg differentiation, promotes T regulatory cell activation or Th17/Treg differentiation; or promotes quorum sensing signaling inhibitors.

29. The method according to claim 24, wherein the inflammatory-related gastrointestinal disorder is an inflammatory bowel disease, wherein the inflammatory bowel disease is selected from the group consisting of: colitis, ulcerative colitis, chronic enteropathy, Crohn's disease and pouchitis; or wherein the inflammatory-related gastrointestinal disorder is food responsive diarrheal disease.

30. The method according to claim 24, wherein the inflammatory-related gastrointestinal disorder is ulcerative colitis, Crohn's disease or food responsive diarrhea.

31. The method according to claim 24, wherein the probiotic composition increases the concentration or expression of an anti-inflammatory cytokine.

32. The method according to claim 24, wherein the subject is a mammal.

33. The method according to claim 24, wherein the subject is a human or companion animal.

34. The method according to claim 24, wherein each probiotic in the probiotic combination is administered to the subject in an amount equating to 10⁸ to 10¹² cfu per day.
